(19) United States
(12) Patent Application Publication
(10) Pub. No.: US 2017/0304621 A1
(43) Pub. Date: Oct. 26, 2017

(54) METHOD AND DEVICE FOR STIMULATING MYELINATED AND UNMYELINATED SMALL DIAMETER VAGAL NEURONS

(71) Applicants: INSTITUT NATIONAL DE LA RECHERCHE AGRONOMIQUE (INRA), Paris (FR); AXONIC, Vallauris (FR)

(72) Inventors: Charles-Henri Malbert, Bruz (FR); Jean-Louis Divoux, Cagnes Sur Mer (FR); David Guiraud, Montpellier (FR); David Andreu, Montarnaud (FR)

(21) Appl. No.: 15/513,166

(22) PCT Filed: Sep. 23, 2015
(86) PCT No.: PCT/IB2015/057336

§ 371 Date: Mar. 22, 2017

Related U.S. Application Data
(63) Continuation-in-part of application No. PCT/IB2014/002210, filed on Sep. 23, 2014.

(51) Int. Cl. A61N 1/36; A61N 1/05

(52) U.S. Cl. CPC A61N 1/36053; A61N 1/36178; A61N 1/0551; A61N 1/36071; A61N 1/36085

(57) ABSTRACT

A method for stimulating vagal neurons as demonstrated by generation of action potentials on these same neurons, wherein electrical pulse trains are periodically applied to electrodes implanted on the anterior and posterior vagus nerve at an entrance of a diaphragm, wherein each electrical pulse train is formed by a plurality of monophasic pulses having a frequency of at least 13.0 kHz.

The present invention concerns a method for stimulating vagal neurons to trigger action potentials on small diameter myelinated A∂ fibers and unmyelinated C fibers.

The vagus nerve is primarily an afferent nerve since the majority of its axons projects from the periphery towards the brain (Grundy, D. “Neuroanatomy of visceral nociception: vagal and splanchnic afferent.” Gut, 51(Supplement 1), i2-i5. doi:10.1136/gut.51.suppl_1. i2, 2002). At the abdominal level, these afferent axons include either myelinated A∂ fibers or unmyelinated C fibers. On the contrary, at the cervical level, Aβ or B type fibers have been described (Duclaux, R., Mei, N., & Ranieri, F. “Conduction velocity along the afferent vagal dendrites: a new type of fibre.” The Journal of Physiology, 260(2), 487-495, 1976).

Electrical vagal nerve stimulation has been used either at the cervical and abdominal level as a potential cure for eating disorders and mainly obesity (McClelland, J., Bozhilova, N., Campbell, I., & Schmidt, U. “A systematic review of the effects of neuromodulation on eating and body weight: evidence from human and animal studies.” European Eating Disorders Review: the Journal of the Eating Disorders Association, 21(6), 436-455. doi:10.1002/erv.2256, 2013). However, this meta-analysis shows that only limited voltage/intensity was used during chronic stimulation: the maximum intensity being no more than 2.5 mA. This intensity converts into a tension of 2.5 Volts for average impedance of the electrode close to the vagus around 1 kOhm. Theses values while protecting the nerve against potentials occurring within the water window (Merrill, D. R. “The Electrochemistry of Charge Injection at the Electrode/Tissue Interface.” In Implantable Neural Prostheses 2 (pp. 85-138). New York, N.Y.: Springer New York. doi:10.1007/978-0-387-98120-8_4, 2010), they are well below the threshold to activate C fibers or small diameter A∂ fibers (Duclaux et al., 1976), (Chen, S. L., Wu, X. Y., Cao, Z. J., Fan, J., Wang, M., Owyang, C., & Li, Y. “Subdiaphragmatic vagal afferent nerves modulate visceral pain.” AJP: Gastrointestinal and Liver Physiology, 294(6), G1441-G1449. doi:10.1152/ajpgi.00588.2007, 2008). As a consequence, while a significant amount of the vagus is likely to be activated during unilateral cervical stimulation such as the one proposed for epilepsy therapy, it is quite likely that only an extremely small fraction of vagal neurons were involved during bilateral subdiaphragmatic stimulation. Nevertheless, a careful review of the bibliography in animal models of chronic vagal stimulation demonstrates that weight loss and/or reduced food intake did exist only when abdominal vagal trunks were stimulated. Experiments reported by Gil et al (2011) and Banni et al (2012) in the rat were enable to exemplify a significative effect over the entire duration of the test period. This contrasted with Matyja et al (2004), Sobocki et al (2006), Biraben et al (2008) and Val-Laillet et al (2011) who find that abdominal VNS was able to permanently reduce weight loss and/or food intake once such effect was observed 2 to 3 weeks after the onset of stimulation.

In theory the very short duration/high frequency of our pulses were unable to create action potentials. High frequency alternating current has been investigated as a solution to modulate vagal activity (Waataja, J. J., Tweden, K. S., & Honda, C. N. “Effects of high-frequency alternating current on axonal conduction through the vagus nerve.” Journal of Neural Engineering, 8(5), 056013. doi:10.1088/1741-2560/8/5/056013, 2011). Using 5 kHz current pulses of 90 μs duration, Waataja and colleagues were able to block the conduction of the vagal nerve as demonstrated by the annihilation of the compound action potential elicited by monophasic pulses applied distally. However, strange behaviour in excitability at frequencies above 12.5 kHz has been observed according to the same model (Rattay, F. “High frequency electrostimulation of excitable cells.” Journal of Theoretical Biology, 123(1), 45-54. 1986). This behaviour generating action potential as if applied to itself has never been tested in experimental practice.

It is an object of the present invention to provide an improved method and device for stimulating myelinated and unmyelinated small diameter vagal neurons such as myelinated A∂ fibers and unmyelinated C fibers suitable for implanted stimulator device. This object is achieved by a method as claimed in claim 1.

To this end, the invention relates to a method for stimulating vagal neurons as demonstrated by generation of action potentials on these same neurons, wherein electrical pulse trains are periodically applied to electrodes implanted on the anterior and posterior vagus nerve at an entrance of a diaphragm, wherein each electrical pulse train is formed by a plurality of monophasic pulses having a frequency of at least 13.0 kHz.

Thanks to the invention, the method allows to effectively activate C fibers and small diameter A∂ fibers while protecting the electrode and the nerve from the water window. Furthermore, because of the reduced power consumption, this invention is suitable for implanted stimulator device with preservation of battery life. This invention is primarily directed towards a cure for eating disorders. Moreover, it is possible to use this invention in the treatment of chronic visceral pain and others disorders.

According to other advantageous aspects of the invention, the method comprises one or more of the following features taken alone or according to all technically possible combinations:
the pulses of each electrical pulse train have constant amplitudes in a period of each electrical pulse train;
the pulses of each electrical pulse train have amplitudes gradually increasing up to a maximum amplitude in a period of each electrical pulse train;
the maximum amplitude of the pulses of each electrical pulse train is a constant current of 10 milliamperes or more;
the maximum amplitude of the pulses of each electrical pulse train is a tension of 10 volts or more;
each electrical pulse train has a duration of 1 millisecond;
each electrical pulse train is applied to myelinated A∂ fibers or unmyelinated C fibers.

The invention also relates to a device for stimulating vagal neurons, the device comprising:
a pulse generator adapted to be implanted and to produce electrical pulse trains; and
a plurality of electrodes adapted to be implanted on the anterior and posterior vagus nerve at an entrance of a diaphragm, the electrodes further structurally adapted to be electrically connectable to the pulse generator for delivering the electrical pulse trains produced by the pulse generator to the anterior and posterior vagus nerve;

characterized in that the pulse generator generates electrical pulse trains each formed by a plurality of pulses having a frequency of at least 13.0 kHz.

The surgical methodology for implanting the device according to the invention or for vagus nerve stimulation is well known to one of skill in the art and may follow that described e.g. by S. A. Reid (“Surgical technique for implantation of the neurocybernetic prothesis.” Epilepsia 31:S38-S39, 1990) for epilepsy treatment. Preferably, the device is implanted under the left hypochondrium.

The invention will be better understood upon reading of the following description, which is given solely by way of example and with reference to the appended drawings, in which:

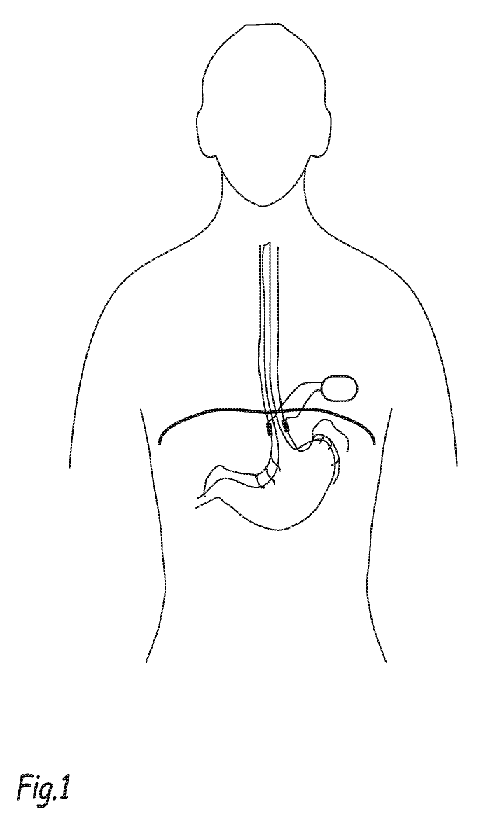

FIG. 1 is a simplified partial front view of a mammal body and of the implanted stimulator device for ventral and dorsal vagus stimulation;

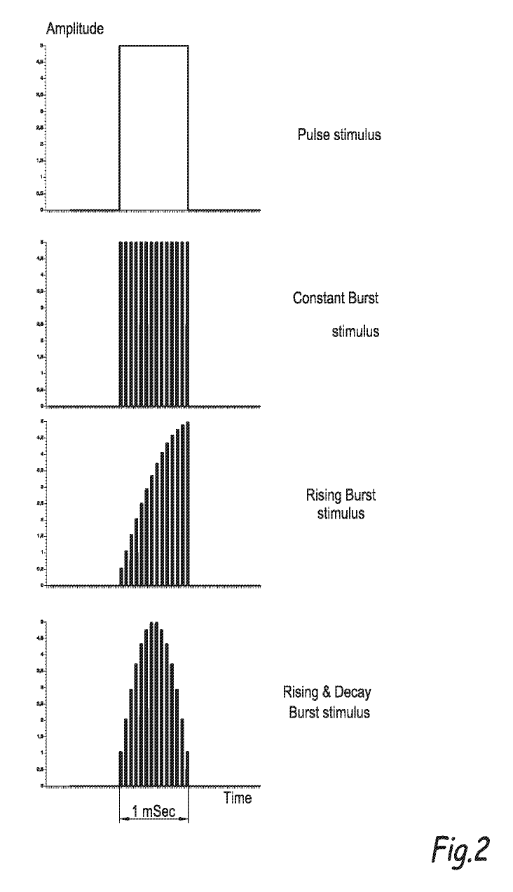

FIG. 2 is a schematic timing chart illustrating four types electrical pulse trains as stimulation schemes;

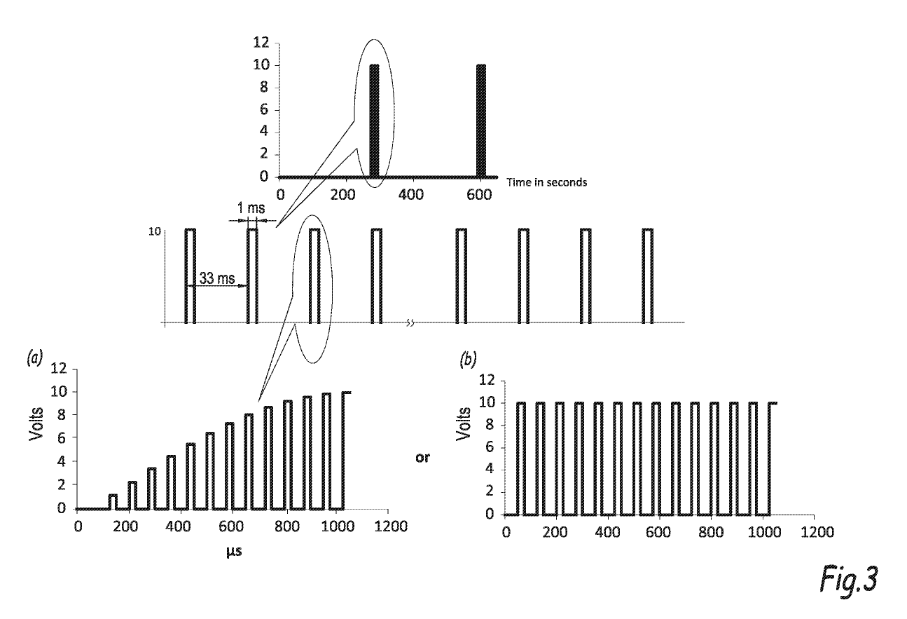

FIG. 3 is a conceptual diagram indicating an example of applying periodical electrical pulse trains;

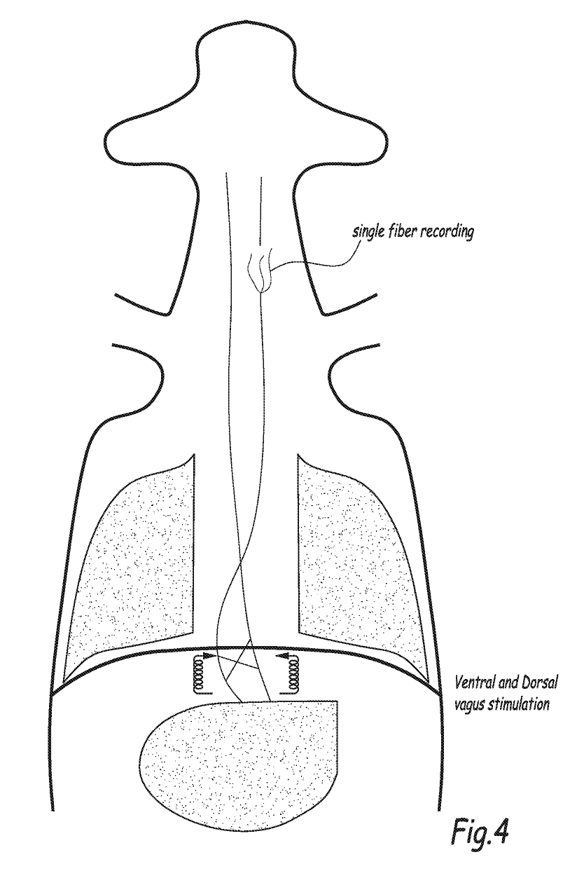

FIG. 4 is a conceptual diagram of an implanted stimulator device for applying current pulses on the anterior and posterior vagus nerve.

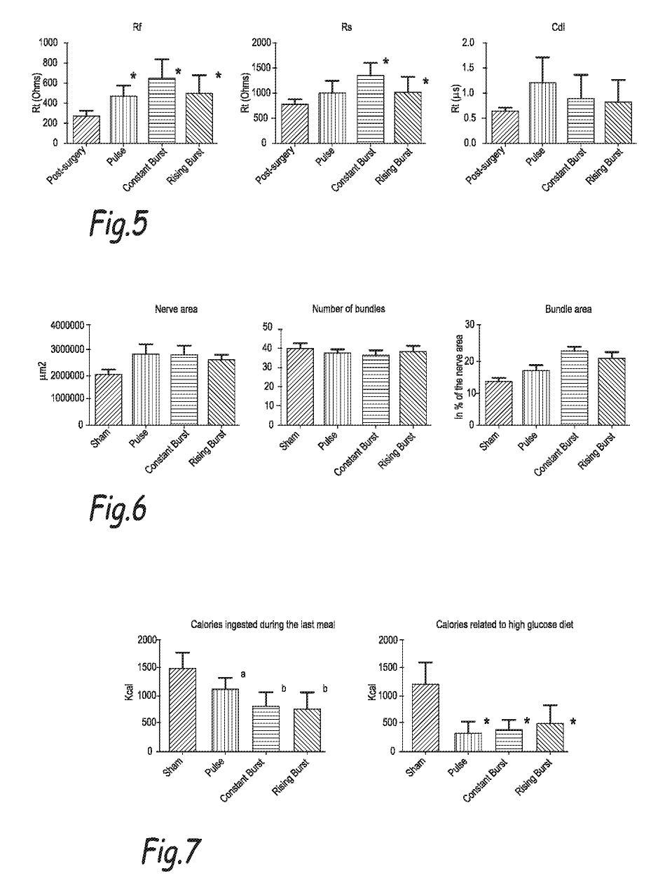

FIG. 5 is a bar graph showing changes in parallel and in series resistance together with associated alternation in parallel capacitance.

FIG. 6 is a bar graph showing quantitative analysis of the area of the nerve, the number of bundles within the nerve and the total areas of these bundles relative to the area of the nerve.

FIG. 7 is a bar graph showing changes in calories ingested and dietary pretences induced by the different patterns of vagal stimulation.

Below, an embodiment of method and device for stimulating myelinated and unmyelinated small diameter vagal neurons pertaining to the present invention will be described using FIG. 1 to FIG. 3.

FIG. 1 shows a simplified partial front view of a mammal body and of an implanted stimulator device for ventral and dorsal vagus stimulation. The implanted stimulator device performs vagus nerve stimulation by applying electrical pulse trains periodically to the ventral vagus nerve (which innervates in part the stomach, the liver and the proximal duodenum) and the dorsal vagus nerve (which innervates in part the stomach and gets lost in the celiac ganglia). Here, the expression “vagus nerve” designates the cranial nerve X and its various branches.

Specifically, the implanted stimulator device includes a pulse generator adapted to produce electrical pulse trains and a plurality of electrodes adapted to be implanted on the anterior and posterior vagus nerve at an entrance of a diaphragm.

The electrodes are structurally adapted to be electrically connectable to the pulse generator for delivering the electrical pulse trains produced by the pulse generator to the anterior and posterior vagus nerve. Each electrical pulse train produced by the pulse generator is formed by a plurality of pulses having a frequency of 13 kHz or, in a variant, higher.

The pulses of each electrical pulse train may have constant amplitudes in a period of each electrical pulse train. Alternatively, the pulses of each electrical pulse train may have amplitudes gradually increasing up to a peak value (maximum amplitude) in a period of each electrical pulse train.

FIG. 2 shows a schematic timing chart illustrating four types electrical pulse trains as stimulation schemes. In this case, the entire duration of each electrical pulse train is 1 mSec as shown in FIG. 2.

First type of the pulse patterns is a “pulse stimulus” from prior art, being at a high voltage state during the entire duration of 1 mSec. Second type of the pulse patterns is a “constant burst stimulus” formed by a plurality of high frequency pulses intermingled with no stimulation episodes in the period. Third type of the pulse patterns is a “rising burst stimulus” having amplitudes gradually increasing up to a peak value (maximum amplitude) in the period. Fourth type of the pulse patterns is a “rising and decay burst stimulus” having amplitudes increasing up to a peak value (maximum amplitude) and decreasing toward zero in the period. Rising and decreasing part of the burst can be, but not limited to, a portion of a sinusoidal, trapezoidal or exponential waveform.

As described in the example later, an experiment was performed by comparing these four types electrical pulse trains as stimulation schemes. The pulse generator in the implanted stimulator device as the present invention may produce at least one of the electrical pulse patterns of the “constant burst stimulus” and the “rising burst stimulus” at a frequency of 13 kHz or higher. As it will be shown later, the “rising burst stimulus” is the more efficient for triggering action potentials on small diameter myelinated A∂ fibers and unmyelinated C fibers.

The present invention triggers action potentials on small diameter myelinated A∂ fibers and unmyelinated C fibers using large current/voltage monophasic pulses of extremely short duration to preserve the nerve and electrodes from damage and to allow stimulation with implanted stimulator. Therefore, the maximum amplitude of the pulses of each electrical pulse train produced by the pulse generator in the implanted stimulator device may be a current of 10 milliamperes or more. In this case, the pulse generator is a current generator, and current signals are applied to the vagus nerves. Alternatively, the maximum amplitude of the pulses of each electrical pulse train produced by the pulse generator in the implanted stimulator device may be a tension of 10 volts or more. In this case, the pulse generator is a voltage generator, and voltage signals are applied to the vagus nerves. In addition, each electrical pulse train has a period of 1 millisecond in this embodiment.

FIG. 3 shows a schematic timing chart illustrating how the high frequency pulses might be incorporated into a more complex scheme suitable for chronic vagal stimulation as described in the PCT application (WO 2009/027425). For example, FIG. 3(a) shows a “burst rising scheme”. FIG. 3(b) shows a “constant Burst scheme” which corresponds to the “constant Burst stimulus” in FIG. 2 in the case of using a voltage generator with a maximum amplitude of 10 volts.

As conditions for all three types schemes in the FIG. 2, on duration of each pulse is 25 μS, and off duration is 50 μS. Therefore, the frequency of the pulses is 13.3 kHz. Duration of the entire train is 1 mSec. As stated above, the pulse generator in the implanted stimulator device 20 as the present invention may produce at least one of the electrical pulse patterns of the “burst rising tension scheme” and the “burst constant tension scheme” in the FIG. 3.

Next, the operations of the implanted stimulator device will be described.

FIG. 3 shows a conceptual diagram indicating an example of applying periodical electrical pulse trains by the implanted stimulator device.

The entire 1 mSec pulse train could be followed by a charge recovery period similar to that often used in classical pulse stimulations. The stimulation by periodical electrical pulse trains lasts 30 seconds, then non-stimulation period lasts 5 minutes.

In this manner, the implanted stimulator device makes it possible to reduce as much as possible the amount of energy applied to the nerve while maintaining the triggering of action potential by these stimulation schemes. Furthermore, the present invention makes it possible to easily trigger action potentials on small diameter myelinated A∂ fibers and unmyelinated C fibers and preserve the nerve and electrodes from damage by using large current/voltage monophasic pulses of extremely short duration. Accordingly, the invention can contribute to a cure for eating disorders. Furthermore, since previous work in a murine model has demonstrated that vagal stimulation at the sub-diaphragmatic level was able to modulate visceral pain (Chen et al., 2008), it is possible to use the present invention in the treatment of chronic visceral pain.

The invention will be further exposed with the following non-limitative example.

### EXAMPLE

### Methods

Electrophysiological experiments were performed on 5 pigs (32±4 Kg, Large White). The experimental procedure was conducted in accordance with the current ethical standards of the European and French legislation (Agreement number A35-622 and Authorization number 01894). The Ethics Committee validated the procedures described in this document (R-2012-CHM-03). The experiment consists in recording evoked action potentials at the cervical level of the left vagal nerve after careful micro-dissection of the nerve bundle to obtain single action potential. Evoked action potentials are generated by applying current pulses on cuff electrodes chirurgically implanted on the anterior and posterior vagus nerve at the entrance of the diaphragm. (FIG. 4)

### Animals and Experimental Set-Up

The animals were pre-anesthetized with Ketamine (5 mg·kg-1 intramuscularly). Suppression of the pharyngo-tracheal reflex was obtained by inhalation of halothane (5% v/v by a face mask) immediately before intubation. A venous cannula was inserted into the marginal vein of the ear to infuse a mixture of a chloralose (60 mg·kg-1, Sigma) and urethane (500 mg·kg-1, Sigma): the primary aesthetic agent. At the completion of the thoracic and cervical surgical procedures, the surgical anaesthesia level was maintained by continuous IV infusion of pentobarbital (20 mg·kg·hr-1, Sanofi). Motion artefacts were cancelled by supplemental slow IV bolus injections of D-tubocurarine (0.2 mg·kg-1, Sigma) every two hours. The surgical level of anaesthesia was continuously assessed by arterial blood pressure measurement obtained from a catheter located in the right carotid artery. The animals were artificially ventilated by a positive pressure ventilator (Siemens, SAL 900) connected to the tracheal cannula. SpCO₂ and O₂ saturation were controlled for normocapnia and SapO₂ at 98% or above using a capnometer connected to the ventilator and a pulse oxymeter placed on the tail of the animal. FiO₂ ranged from 30 to 45%. Body temperature was kept at 38.5±0.5° C. by a self-regulating heating element placed under the animal.

At the end of the experiment, the animals were killed by an overdose of pentobarbital IV.

### Design of Stimulating Electrodes and Vagal Placement

The stimulating electrodes consisted in cuff electrodes for a nerve diameter target of 3.0±0.1 mm. They comprised two pairs of Pt-Ir10% half circular contacts (4 in total), short-circuited together to form a bipolar configuration. Each pair of contacts is situated on both sides of a tube, forming a circumference, and 10 mm distant from the other pair of contacts. The overall dimension of the tube is 25±0.1 mm to provide the electrode with proper insulation from the surrounding environment. A 0.1 mm recess from the contacts to the surface of the nerve is provided to avoid direct interaction between metal and living tissues. The electrode device is realized by means of overmolding the set of contacts, using a high consistency rubber silicone of long-term implantable medical grade. The assembly is armoured with polyester mesh that also serve as fastening the device by means of clipping.

Both poles of the electrode are output by means of flexible, polyester insulated, multi-strands, medical grade stainless steel cables embedded in dedicated implantable grade rubber silicone bilumen tubing.

A surgical access to the mediastinal area was achieved at the level of the 8^{th} intercostal space while the animal was in right lateral decubitus. The vagal trunks were dissected over 5 cm as close as possible to the entrance of the diaphragm to by-pass the interconnections between the dorsal and ventral trunks present posterior to the heart. The cuff electrodes were placed around both vagal trunks and maintained closed by stiches on the proximal and distal end of the Dacron covered cuffs. The pressure on the vagus nerve was selected for an adequate closure of the cuff while maintaining its ability to move up and down alongside the nerve.

### Impedance Measurement

At the end of the recording procedure and immediately before euthanasia, impedance of the stimulating electrodes was recorded according We et M Grill (Wei, X. F., & Grill, W. M. “Impedance characteristics of deep brain stimulation electrodes in vitro and in vivo.” Journal of Neural Engineering, 6(4), 046008. doi:10.1088/1741-2560/6/4/046008, 2009) using purpose made stimulating and recording device controlled with dedicated software written under Labview 2011 (National Instrument, USA). The current stimulator was able to generate 1 ms current pulses from 0.1 to 2.5 mA amplitude and was fully insulated. The amplifier connected in parallel to the stimulator output consisted in a NI USB 621 card and was also isolated from the remaining equipment. A total of 20 pulses with a amplitude step of 0.1 mA was performed and analysed with a Randles equivalent circuit with a Warburg impedance negligible. The impedance used for current calculation in the remaining part of this paper corresponded to the mean value of impedance against current while the curve was stable. (mainly between 1 to 2 mA).

### Pulses Generation

Pulses generation was performed either in voltage or current configuration.

For voltage configuration, a digital to analogue card (National Instrument, USA) coupled with a dedicated software writing under Labview 2011 was used to generate the pulse pattern together with the synchronised trigger pulse used for data acquisition. Four pulses patterns could be generated every 2 Hz. They are summarized in FIG. 2. The voltage output of the D/A card was connected to a buffer amplifier adapted for the impedance of the vagal trunks. The buffer amplifier was insulated from the remaining part of the electronic circuitry by optocoupling and the power supply was achieved by the means of rechargeable batteries. The second output of the D/A used to generate the trigger pulse at the onset the pulse pattern was hocked to the trigger input of the A/D card.

In current configuration, the pulses are generated in 3 different modes: classical rectangular active pulse with an amplitude and a pulse width of respectively 2.5 mA and 1 ms; burst of rectangular pulses, 15 mA 50 μs pulse width separated by 75 μs of high impedance for a total duration of 1 ms; the same burst but with a one fourth sinus rising envelope.

### Vagal Recordings

Electrical activity from single vagal afferent neurons was recorded by classical neurophysiological methods adapted to the pig. Briefly, the left vagus was made free from surrounding connective tissue. The skin and cervical muscles were sutured to a metallic frame to create a pool filled with warm paraffin oil. Monopolar recordings of vagal bundles were performed after section of the cervical vagus and micro-dissection of its distal end. Adequate amplification of the signal was provided by a homemade amplifier (gain 50000, impedance 20 Mohms), placed near the recording electrodes (tungsten, 50 μm, WPI USA). After low and high pass filtration (300-6000 Hz), the raw electroneurogram was stored on a hard drive following Analog to digital conversion at 20 KHz performed using a build in house software written under Labview 2011 (National Instruments, USA). Unitary vagal activity was discriminated off-line using adaptive shape matching criteria.

Recording of evoked potential was performed on the same computer with different software dedicated to single fibre evoked potential recording. The AD card was set-up in a double-buffered triggering configuration so that the rising edge of each trigger pulse generated in synchrony with stimulating pulse was able to launch an acquisition sweep lasting 500 mSec. The acquisition frequency of this sweep was 40 KHz. The recurrence of each sweep was 2 Hz to avoid collision along the nerve between the stimulation and recording site (30 cm). This configuration is therefore able to discriminate neurons with conduction speed well below 1 m/Sec.

Evoked potential was performed on well characterized gastric or duodenal projecting afferent neurons only. Therefore prior to vagal stimulation, via trials and errors, we were looking for a neuron included in a nerve bundle that increased significantly its firing frequency during light distension of either the stomach or the duodenum. To achieve theses distensions, a mid-line laparotomy was performed prior to nerve dissection in order to insert inflatable balloons in the stomach and in the duodenum. A double-lumen catheter (ID 3.5 mm for air injection/retrieval and ID 1.0 mm for pressure sensing) incorporating a 15 cm-long latex balloon was placed in the proximal duodenum immediately after the pylorus. The oral end of the catheter was transmurally sutured to the gut in order to avoid movement of the balloon into the stomach. The larger-bore opening was used for air injection and retrieval, allowing inflation and deflation of the latex balloon. The smaller-diameter opening was connected to a pressure transducer (PX23, Gould) to record the static air pressure within the balloon in the absence of artefacts related to the dynamic pressure changes during inflation and deflation. The same set-up was used for the gastric balloon made off a one-litter silicon spherical bag. Rapid balloon distension of the duodenum or the stomach was used to identify mechanosensitive units. This was achieved by connecting one of each balloon to a compressed air source (750 mmHg) through a computer-controlled valve until the pressure within the balloon equalled 20 mmHg. Thereafter, the balloon was deflated by computer-controlled connection of the balloon to a vacuum source (−75 mmHg).

### Data Analysis

Evoked potential analysis was performed using dedicated software written in the laboratory under Labview. This software allows following the occurrence or the absence of action potential in three dimensions: time of occurrence during the sweep, sweep number and amplitude of the action potential. The conduction speed was automatically calculated knowing the time of occurrence of the action potential long the sweep and the distance between the stimulating and recording electrodes.

We found extremely difficult to evaluate the distance between recording and stimulating electrodes by the means of a necropsy. Therefore, at the end of the experiment, the animal was placed under a CT (Hi-Speed, GE, USA) to calculate this distance within a centimetrer resolution. A whole body helicoidal scan was performed from the last thoracic vertebra up to the head with millimetre thick slice after reconstruction. The images were transferred to Osirix software (Rosset, Spadola, & Ratib, 2004). A three dimensional reconstruction was performed from the individual transaxial slices and using the adequate tool in Osirix, the distance between the stimulation electrodes and the recording site calculated for each animal.

### Results

### Identified Neurons and Area of Projection

A total of 15 slow adapting mechanosensitive neurons were identified. Four of them have their receptor field located in the duodenum while the remaining 11 have their receptor field located in the stomach. Half adaptation time equalled 4.3±0.08 sec for the duodenal projecting neurons and 3.2±0.04 sec for the gastric ones. The firing threshold of the gastric neurons was higher than the duodenal ones: 18±3.1 mmHg vs 20±2.8 mmHg respectively.

### Impedance of Stimulating Electrodes

The impedance of the stimulating electrodes was remarkably stable between animals: 986±83 Ohms. There was no significant difference between the impedance of the anterior and posterior vagus nerve. The impedance data were used afterwards for calculation of the amount of injected electrical charges in voltage stimulation Mode.

Voltage pulses

Voltage pulses were tested on two animals only while current pulses were used for the remaining animals. The voltage threshold to generate an action potential was obtained by sequential increase in voltage applied in parallel on both electrodes. Conduction speed was calculated immediately afterwards. The voltage threshold to generate the same action potential was also calculated for each of the burst type procedure applied at random. Data are presented in Table 1.

TABLE 1
Charges injection threshold for triggering an action potential depending on the shape
of the stimulating pulses. Stimulation is performed in voltage mode. Pulse stimulus
was set to 1 msec, the pulses within the burst are set to 25 μsec on and 50 μsec off
and the entire burst lasted 1 msec. Conduction speed was calculated with pulse type
stimulus. Neuron 2 and 3 were found on the same animal and on the same vagus.

| | | | | | | | Rising |
|---|---|---|---|---|---|---|---|
| | | | | | Constant | Rising | and decay |
| | Conduction | | | Pulse | burst | burst | burst |
| | speed | Receptive | Impedance | stimulus | stimulus | stimulus | stimulus |
| Neuron | (m/s) | field | (Ohms) | (μC) | (μC) | (μC) | (μC) |
| 1 | 4.5 | Stomach | 950 | 19 | 7.1 | 5.1 | 14.1 |
| 2 | 2.3 | Stomach | 1020 | 21.2 | 8.6 | 5.98 | 16.2 |
| 3 | 5.1 | Stomach | 1020 | 16.8 | 6.2 | 4.61 | 13.4 |
| 4 | 2.6 | Duodenum | 985 | 21.7 | 8.4 | 5.62 | 18.8 |

Rising burst stimulus was the most effective method to trigger action potential irrespective of the nature of the neuron or its conduction speed. The amount of charges required for activating a neuron was about ⅓ of that observed for classical pulse pattern. Surprisingly, the rising and decay burst stimulus was almost ineffective to trigger action potential. Knowing that the shape of the burst as an important issue, we wanted to know how important was the frequency of each single burst within the pulse. Therefore we investigate the potency to generate action potential during different combinations of pulse duration within the burst as well as the duration of the non-stimulation period during the pulse.

Three pulse durations were tested during the pulse while having the inter-pulse duration fixed at 50 μS: 25, 80 and 150 μS ending with a stimulation frequency of 13.3, 7.7 and 5.0 KHz respectively. To cancel the changes in charges input, the number of pulses within the burst was also changed so to have for constant pulse stimulation scheme a total charge of 0.3 μC/volts. Therefore the stimulation frequency of 13.3; 7.7 and 5.0 KHz were used for 14; 4 and 2 pulses respectively. While the 13.3 KHz frequency was able to trigger action potential as indicated in table 1, we were not able to generate action potential with the other frequency tested irrespective of the tension applied at the electrode (within the limits of the generator i.e. up to 30 Volts).

### Current Pulses

Data obtained from current stimulation confirmed those acquired in voltage mode. The most effective solution for stimulating C or A∂ gastric or duodenal afferent neurons was a rising burst stimulus (Table 2) for pulses lasting 25 μs at a frequency of 13.3 KHz.

TABLE 2
Charges injection threshold for triggering an action potential depending on the
shape of the stimulating pulses. Stimulations were performed in current mode.

| | | | | | | | Rising |
|---|---|---|---|---|---|---|---|
| | | | | | Constant | Rising | and decay |
| | Conduction | | | Pulse | burst | burst | burst |
| | speed | Receptive | Impedance | stimulus | stimulus | stimulus | stimulus |
| Neuron | (m/s) | field | (Ohms) | (μC) | (μC) | (μC) | (μC) |
| 1 | 2.3 | Stomach | 965 | 20.4 | 8.6 | 5.9 | — |
| 2 | 2.3 | Stomach | 965 | 20.4 | 8.0 | 5.5 | — |
| 3 | 2.4 | Stomach | 1175 | 18.1 | 6.2 | 4.3 | 19.3 |
| 4 | 6.8 | Stomach | 1023 | 16.0 | 5.6 | 3.9 | 16.5 |
| 5 | 3.5 | Stomach | 995 | 16.2 | 5.5 | 4.1 | 18.3 |
| 6 | 4.8 | Stomach | 995 | 17.5 | 6.7 | 4.8 | 17.5 |
| 7 | 3.9 | Stomach | 893 | 16.6 | 6.2 | 4.3 | 18.4 |
| 8 | 4.0 | Stomach | 893 | 18.2 | 6.4 | 4.7 | 18.8 |
| 9 | 2.3 | Duodenum | 1175 | 18.9 | 7.2 | 5.0 | — |
| 10 | 2.9 | Duodenum | 995 | 16.2 | 5.7 | 4.0 | — |
| 11 | 2.9 | Duodenum | 1022 | 18.1 | 6.8 | 4.9 | 18.5 |

(—) Unable to trigger action potential at the maximal current supplied by the stimulating device.

### Supplementary Material

### Aims

Evaluate the use of the most effective stimulating patterns on conscious animals in a chronic experimental paradigm (8 days) with specific reference to feeding behavior.

### Animals and Experimental Set-Up

Four groups of six growing pigs each were used for this experiment (32±4.4 kg). The French government under the reference 00341.01 approved this experiment on the 21 Nov. 2013.

Each group received either no stimulation (sham group), pulse stimulation, constant burst stimulation or rising burst stimulation all of them being in current mode. The detailed characteristics of these stimulations/groups were described in the Pulses generation section. The Rising and Decay burst stimulation, described in the initial patent, was not used in this experiment since it appears to be the least effective pattern capable to trigger action potential in anesthetized animals.

The experiment consists in placing under laparoscopy two cuff electrodes on the anterior and posterior vagal trunks at the level of the lower oesophageal sphincter. The wires of these electrodes were tunneled under the skin up to the interscapular area where they were immediately connected to a dedicated portable neurostimulator capable to generate on a permanent basis pulse, constant burst or rising burst stimulation profiles. For the sham group a dummy box was connected to the electrodes. The animals were allowed to recover from the minimally invasive surgery during one day after which the stimulator was started at the required current. The impedance of the electrodes was also checked using purposely-designed device at this stage. Three days after the onset of stimulation or four days after the surgery for the sham group, the animals were submitted to a multiple choice eating behaviour test investigating the impact of vagal stimulation on food intake pattern. This test was continued until day 8 post stimulation. The animals were imaged at this time (not shown) and euthanized afterwards to sample the vagus nerve for histological analysis.

### Design of Stimulating Electrodes

The stimulating electrodes consisted in cuff electrodes for a nerve diameter target of 3.0±0.1 mm. They comprised two pairs of Pt—Ir 10% half circular contacts (4 in total), short-circuited together to form a bipolar configuration. Pairs of contacts were located on both sides of a tube, forming a circumference, and 10 mm distant from the other pair of contacts. The overall dimension of the tube was 25±0.1 mm to provide the electrode with proper insulation from the surrounding environment. A 0.1 mm recess from the contacts to the surface of the nerve was provided to avoid direct interaction between metal and living tissues. The electrode device was build by means of overmolding the set of contacts, using a high consistency rubber silicone of long-term implantable medical grade. The assembly was armoured with polyester mesh that also serve as fastening the device by means of clipping.

Both poles of the electrode were exited by means of flexible, polyester insulated, multi-strands, medical grade stainless steel cables embedded in dedicated implantable grade rubber silicone bilumen tubing.

### Vagal Electrodes Placement

Two days before the surgery, the animals received exclusively a low residue meal consisting in a high protein liquid diet (Clinutren 1.5) so to clear the stomach from food particles. Additional drainage was performed immediately before surgery and after tracheal intubation by inserting a drainage tube down to the stomach with endoscopic guidance. This tube was left in place during approximately the first half of the surgical procedure.

Vagal electrodes placement was performed under general anaesthesia achieved by inhalation of isoflurane supplied a positive pressure ventilator (AS/3, General Electric) to the tracheal cannula and by IV infusion of Fentanyl (7 μg/kg/min). The anaesthesia level and tidal volume were set and vital signs continuously monitored so to maintain a Minimum alveolar concentration of isoflurane of 2.0, a SaPO2 not less than 97% and a saPCO2 between 4.5 and 5%. Arterial pressure and ST segment were also monitored.

The stimulating electrodes consisting in two cuffs were implanted laparoscopically. Device implantation by the experienced surgeons typically took 60 to 90 minutes; 5 ports were used including the camera port. The implantation was performed with the pig in right decubitus so to expose the crus and the gastro-esophageal junction.

Intra-abdominal dissection and electrode placement were accomplished in the following sequence. The hepatophrenic ligament was dissected on its top part to expose the anterior gastro-esophageal junction. The stomach was pulled backward to keep slight tension on the gastro-esophageal junction and to remove the spleen from the field of view. The lesser omentum is dissected along side the esophagus from the diaphragmatic hiatus down to the lower part of the lower esophageal sphincter so to exposed about 8 cm of esophagus. The oesophagus was afterward reclined to expose and dissect the posterior vagus trunk over about 5 cm using a right-angled dissector (Microfrance CEV501). The same was performed for the anterior vagus trunk. In some animals, a small vagal branch originates from the distal part of anterior vagus and reached to proximal part of the posterior vagus. Since this branch limits the length of accessible anterior vagus, we decided to cut this branch on all animals irrespective of its experimental group. One cuff is placed afterwards under the posterior vagus and lifted by a grasper holding the Dacron flaps so to locate the vagal trunk inside the groove of the cuff. It was fixed in position by a surgical titanium clip (Ligamax 15 M/L, Ethicon) placed astride both Dacron flaps orally. A second surgical clip was placed aborally also astride the Dacron flaps using a right angle clip applicator (Acuclip OMSA8, Covidien). Once both clips were in position, the surgeon check for a free moving cuff alongside the vagal trunk. The same procedure was performed for the anterior vagus. The omentum was closed afterwards by a V-Loc—Endostitch loaded running suture. A wires loop of 10 to 15 cm was created inside the abdomen so that no strain-reliefs were required to alleviate the physical stress on the connecting wires.

### Vagal Electrodes Impedance

Impedance of the stimulating electrodes was recorded the day after surgery. The evolution of the impedance was checked again 8 days after the onset of the stimulation irrespective of its mode. The method used was derived from We et Mc Grill ¹and it was performed using purpose made stimulating and recording device controlled with dedicated software written under Labview 2011 (National Instrument, USA). The current stimulator was able to generate 1 ms current pulses from 0.1 to 2.5 mA amplitude and was fully insulated. The amplifier connected in parallel to the stimulator output consisted in a NI USB 621 card and was also isolated from the remaining equipment. A total of 20 pulses with an amplitude step of 0.1 mA was performed. The impedance used for current calculation corresponded to the mean value of the impedance against current while the curve was stable (mainly between 1 to 2 mA). ¹ Xuefeng F Wei and Warren M Grill, “Impedance Characteristics of Deep Brain Stimulation Electrodes in Vitro and in Vivo,” Journal of Neural Engineering 6, no. 4 (Jul. 9, 2009): 046008, doi:10.1088/1741-2560/6/4/046008.

Analysis of the current-voltage matrix was performed using dedicated labview software designed to perform a non-linear adjustment of the We et Mc Grill formula based on the Randles equivalent circuit with a Warburg impedance negligible. The non linear fitting was performed using a Levenberg-Marquardt algorithm.

### Pulses Generation

Three types of vagal stimulation were achieved depending on the experimental group. A recovery pulse of opposite value followed the train of burst or the single pulse, depending of the stimulation scheme.

Once started, the stimulation parameters, including the pulse current, were maintained constant for the duration of the experiment. All three stimulation schemes were active during 30 seconds and were inactive during 300 seconds to match the pattern described partially in FIG. 3 of the present application.
Pule stimulation—duration of the pulse—1 ms, frequency of pulses within the trains—30 Hz; duration of the train—30 s; interval between trains—300 s and amplitude of the pulses—2.5 mA).
Constant burst stimulation—Instead of using a long duration 1 ms pulse, they were minced into 14 short lasting current pulses each of them lasting 25 μs and intermingled with no current for 50 μs. The amplitude of these pulses was constant within the burst and set at 15 mA. All the remaining parameters were identical to pulse stimulation scheme.
Rising burst stimulation—This stimulation scheme is identical to constant burst stimulation but the current was not constant during the burst. The current was increased in semi-sinusoidal manner so to reach 15 mA on the last micropulse of the burst.

### Food Intake Behaviour

Pigs received all their food from a robotic feeder comprising three troughs placed side by side. Enclosures were connected to computer running software developed in our laboratory with Labview. This system recorded continuously the amount of food remaining in each trough via a strain gauge located under the trough (acquisition frequency 1 Hz). A low pass filter (0.3 Hz, −40 dB) was used to minimize the artefacts generated by movements of the animal. The system was linear from 0 to 3 kg (±0.01%) and its sensitivity was ±3 g full scale. The recorded raw data were then transferred to another home made software that automatically extracted different parameters necessary to calculate several variables for eating behaviour pattern analysis. The changes in the amount of food remaining in the trough, the number of visits, the time and the duration of each visit, were obtained. Furthermore the following variables were also calculated: total eating duration, amount of food ingested, number of eating bouts, intake speed.

Animals had simultaneous access to the control (balanced), high lipids and high glucose test feeds during 30 minutes at 9H00, 12H30 and 17H00 to assess their food preferences and food intake pattern. Every time a meal was distributed, 300 g of each test feed was placed into the three troughs in a different order per testing day to avoid any bias. The computer software was then activated to allow access to the test feeds. Animals had ad libitum access to water during the whole test. The composition of the test feed was designed so to have close amount of calories per gram despite large changes in composition. The composition of the test feed was given in additional Table 3.

TABLE 3
Composition of the test feed. Data are given for 100 g of feed

| | Control | High glucose | High lipids |
|---|---|---|---|
| Crude proteins (g) | 18.2 | 14.7 | 14.8 |
| Crude fiber (g) | 4.0 | 3.2 | 3.2 |
| Starch (g) | 36.9 | 29.8 | 31.4 |
| Total sugar (g) | 5.0 | 19.1 | 4.1 |
| Fat (g) | 4.0 | 3.2 | 18.3 |
| Energy (kcal) | 332 | 342 | 396 |

### Histology

At the end of the experiment, the animals were euthanatized using T61. Afterwards, a length of 10 cm of the vagus was sampled so to have the stimulating cuff in the sample or the equivalent segment for the sham animals. All the samples were fixed in 4% paraformaldehyde and paraffin-embedded. The paraffin blocks were subsequently cut on a Leica RM2145 microtome to produce 5-μm slices that were stained with hematoxylin-eosin. One slice every 2 mm was used for the microscopic analyses. The nerve section area was digitized at a 100-fold magnification with an Eclipse E400 Nikon microscope and analyzed using ImageJ software.

### Results

### Impedance

Voltage changes during the 1 msecond—1 mA current pulse could be always adjusted using the Wei and McGrill equation except for two samples in the constant burst group and another two samples in the rising burst group. These matrices could not be fitted using the Levenberg Marquadt algorithm since no optimum was found during the fitting process. Electrode impedance followed always an identical pattern irrespective of the stimulation pattern i.e. pulse, constant burst and rising burst stimulations did not differed. Nevertheless, we observed a significant increase in parallel and in serie resistances together with an increased SD for in parallel capacitance without actual significant changes in the mean Cdl.

FIG. 5 shows that changes in parallel and in series resistance together with associated alteration in parallel capacitance. * denotes a significant difference (p<0.05) from post-surgery. The three last bars were obtained 8 days after the data depicted in the left one. The stimulation was stopped a couple of minutes before doing the measurement so to obtain the impedance value.

### Histology of the Vagus

No difference can be found between the dorsal or the ventral vagus. We were not able to identify any significant lymphoid infiltration at or within the vicinity of the cuff. Similarly, no sign of haemorrhage was observed close or within the cuff itself.

Quantitative analysis of the histological samples did not shown a significant difference in the nerve area between groups while there is a tendency to observe an increased nerve section. Similarly, the injection of current on the nerve irrespective of the pattern of application did not alter the number of bundles. However, we found a significant increase in the area of the bundle relative to the nerve size (in %) for the constant burst and the rising burst groups compared to the sham group.

FIG. 6 shows that quantitative analysis of the area of the nerve, the number of bundles within the nerve and the total areas of these bundles relative to the area of the nerve. Data of the dorsal and ventral vagi have been pooled since no differences were found between these. * denotes a significant difference (p<0.05) from data obtained in the sham group.

### Food Intake Pattern

The daily amount of ingested diet did not differed between groups (1603±103.5, 1612±130.3, 1619±141.3 and 1607±148.4 for sham, pulse, constant burst and rising burst respectively). The same feature was also found when the nature of each component of the diet was taken into account with specific reference to the caloric density of control, hyperglucidic and hyperlipidic diets: 5438±350.7, 5543±429.1, 5588±531.4 and 5553±553.3 kcal/day for sham, pulse, constant burst and rising burst respectively.

Since we used growing animals with huge caloric requirements to achieve their growing potential, it is extremely difficult to alter their eating pattern on a daily basis. This is why we concentrate our further analyses on food intake pattern occurring during the last meal of the day that by virtue of the experimental protocol reflect more a pleasurable appetite than the first two that represent an absolute metabolic requirement². Using this new experimental paradigm, we found that the amount of calories ingested drop significantly for constant burst and rising burst groups. Furthermore, it was also reduced for pulse stimulus group but to a lesser extend than the two burst groups (FIG. 7) ² s Guerin et al., “Changes in Intragastric Meal Distribution Are Better Predictors of Gastric Emptying Rate in Conscious Pigs Than Are Meal Viscosity or Dietary Fibre Concentration.,” British Journal of Nutrition 85, no. 3 (March 2001): 343-50, doi:10.1079/BJN2000271.

FIG. 7 shows that changes in calories ingested and dietary pretences induced by the different patterns of vagal stimulation. The last three days of data (D+6, 7 and 8 after the onset of stimulation) were pooled. The last meal of the day representative of the pleasurable appetite was served by a robotic assistant at 17H00 and was programmed to last 30 minutes. (a and b) denotes a significant difference level (0.05 and 0.01 respectively) from sham and pulse. * denotes significant difference from sham only.

The analysis of the individual sampling in each trough showed that all type of stimulation reduced significantly the ingestion of the most preferred diet by the species i.e. high glucose diet. Furthermore, we found that constant burst and rising burst stimulations were also associated with an increased ingestion of high lipids diet in 2 out of 6 of the animals for each group; a pattern never found in pulse or sham group.

### CONCLUSIONS

Despite a relatively short duration of stimulation, we observed a large impact of constant burst and rising burst stimulation patterns on food intake. These changes were far more obvious than the one observed with the more classical pulse stimulation. The larger current injection used for the more efficient stimulation patterns were not more damageable for the nerve than the 2 mA pulse stimulus since neither the number of bundle and the nerve area were altered by the burst type stimulation compared to pulse stimulation. This was confirmed in part by the network analysis of the impedance of the nerve.

Therefore, we have demonstrated that both burst stimulation patterns might represent a more effective alternative to classical pulse stimulation within the scope of reducing food intake. We further demonstrate the capability to use such pattern in a chronic stimulation set-up without major alteration in the nerve structure or its electrical characteristics. Finally, we did not find any differences between constant burst and rising burst patterns that behave in a similar manner.

1. A method for stimulating vagal neurons as demonstrated by generation of action potentials on the vagal neurons, wherein electrical pulse trains are periodically applied to electrodes implanted on anterior and posterior vagus nerve at an entrance of a diaphragm, wherein each electrical pulse train is formed by a plurality of monophasic pulses having a frequency of at least 13.0 kHz.

2. The method according to claim 1, wherein the pulses of each electrical pulse train have constant amplitudes in a period of each electrical pulse train.

3. The method according to claim 1, wherein the pulses of each electrical pulse train have amplitudes gradually increasing up to a maximum amplitude in a period of each electrical pulse train.

4. The method according to claim 1, wherein the maximum amplitude of the pulses of each electrical pulse train is a current of 10 milliamperes or more.

5. The method according to claim 1, wherein the maximum amplitude of the pulses of each electrical pulse train is a tension of 10 volts or more.

6. The method according to claim 1, wherein each electrical pulse train has a duration of 1 millisecond.

7. The method according to claim 1, wherein each electrical pulse train is applied to myelinated A∂ fibers or unmyelinated C fibers.

8. A device for stimulating vagal neurons, the device comprising:
a pulse generator adapted to produce electrical pulse trains; and
a plurality of electrodes adapted to be implanted on the anterior and posterior vagus nerve at an entrance of a diaphragm, the electrodes further structurally adapted to be electrically connectable to the pulse generator for delivering the electrical pulse trains produced by the pulse generator to the anterior and posterior vagus nerve;
wherein the pulse generator generates electrical pulse trains each formed by a plurality of monophasic pulses having a frequency of at least 13.0 kHz.

9. The device according to claim 8, wherein the pulses of each electrical pulse train have constant amplitudes in a period of each electrical pulse train.

10. The device according to claim 8, wherein the pulses of each electrical pulse train have amplitudes gradually increasing up to a maximum amplitude in a period of each electrical pulse train.

11. The device according to claim 8, wherein the maximum amplitude of the pulses of each electrical pulse train is a current of 10 milliamperes or more.

12. The device according to claim 8, wherein the maximum amplitude of the pulses of each electrical pulse train is a tension of 10 volts or more.

13. The device according to claim 8, wherein each electrical pulse train has a duration of 1 millisecond.
